(19) United States
(12) Patent Application Publication
(10) Pub. No.: US 2020/0093413 A1
(43) Pub. Date: Mar. 26, 2020

(54) A PELVIC FLOOR MUSCLE TRAINING AND PROFILING APPARATUS FOR INSERTING INTO A BODY CAVITY OF A HUMAN AND A METHOD FOR VISUALIZING PELVIC FLOOR MUSCLE CONTRACTIONS

(71) Applicant: Novipel Holding APS, Frederiksberg (DK)

(72) Inventors: Alexander Hennings, Kobenhavn NV (DK); Jakob Christian Konradsen, Kobenhavn NV (DK); Noam Ritov Epstein, Birkerod (DK)

(73) Assignee: Novipel Holding APS, Frederiksberg (DK)

(21) Appl. No.: 16/065,264

(22) PCT Filed: Dec. 28, 2016
(86) PCT No.: PCT/DK2016/050473

§ 371 Date: Jun. 22, 2018

Related U.S. Application Data
(60) Provisional application No. 62/271,619, filed on Dec. 28, 2015.

(51) Int. Cl. A61B 5/22; A61B 5/00; A63B 23/20

(52) U.S. Cl. CPC A61B 5/227; A61B 5/4255; A61B 5/4337; A61B 5/04882; A61B 5/6885; A61B 5/742; A63B 23/20; A61B 5/486

(57) ABSTRACT

A pelvic floor muscle training and profiling apparatus for inserting into a body cavity of a human, said apparatus comprising; a body having a distal end, an opposite proximal end and an outer surface, the body extending between said ends, a sensor array encompassed in the body, the sensor array comprising a sensor unit, the sensor unit being adapted for detecting an exterior force applied to the outer surface of the body, a micro-control unit adapted to receive sensor data from the sensor array, wherein the sensor unit is adapted for detecting a specific position of the exterior force applied to the outer surface of the body.

### TECHNICAL FIELD

The present disclosure relates in a first aspect to an apparatus and in a second aspect to a method for visualizing pelvic floor muscle contractions.

### BACKGROUND INFORMATION

One function of the pelvic floor is to support organs that are located in the pelvis area. Urinary and fecal incontinence, prolapses, infections, inflammations and pain in the vaginal and rectum area, decreased sexual pleasure, and other conditions, can all result from dysfunctional pelvic floor muscles. Dysfunction in the pelvic floor muscles can be the result of a variety of factors, including hormonal changes, aging, heavy lifting, excessive coughing, and others.

Exercises employing pelvic floor muscle clenching or the practice of holding weights in the vagina have been developed to assist with improving pelvic floor muscle strength. These methods have limited success, since it is difficult to quantify if the exercise i.e. the muscle clenching is done correctly at all times.

The way a health care provider (HPC) controls if a user performs an exercise correctly, is by inserting a finger in the opening and feeling the clenching around the finger.

Although the HPC may instruct a person on how to correctly perform the exercise in order to improve pelvic floor muscle function, the HPC is not able to provide constant feedback (e.g. when the person practices the method at home), therefore the person runs the risk of practicing the method incorrectly and thereby defeating the purpose of the exercise.

Such an example is given in WO2016/042310, which discloses a device for measuring the presence of a pelvic floor muscle contraction. The device comprises a two-part housing having a body, a force sensing sensor encompassed within the body of the housing, and a data receiver. Upon a contraction of a pelvic floor muscle, the two parts of the housing are pushed towards each other via a slidable finger comprised in the housing, and the force sensor measures the force that said slidable finger exerts on the sensor.

Thus this device only facilitates the possibility of detecting that a force (possibly due to a clenching of a muscle) is exerted on it, but gives no information as to the source of the force or the correctness of the muscle clenching.

It therefore still remains a challenge to effectively measure the contraction of the pelvic floor muscles e.g. levator ani and provide valuable feedback to the user.

### SUMMARY OF THE INVENTION

It is therefore an object of the present invention to provide a pelvic floor muscle training and profiling apparatus which is easy and comfortable to use and improves the quality of the exercises relating to strengthening the pelvic floor muscle.

Furthermore, it is an object of the invention to provide a method for improving the feedback from the apparatus to the user so as to improve the quality of the exercise of the pelvic floor muscle.

In a further object of the invention the apparatus according to the invention is used to visualize the movement of pelvic floor muscle, tissue and/or abscess.

In a first aspect of the invention this is solved by providing a pelvic floor muscle training and profiling apparatus for inserting into a body cavity of a human, said apparatus comprising;

a body having a distal end, an opposite proximal end and an outer surface, the body extending between said ends,

a sensor array encompassed in the body, the sensor array comprising a sensor unit, the sensor unit being adapted for detecting an exterior force applied to the outer surface of the body,

a micro-control unit adapted to receive sensor data from the sensor array,

wherein the sensor unit is adapted for detecting a specific position of the exterior force applied to the outer surface of the body.

By determining the position of a force applied on the outer surface of the body, the apparatus can provide a more detailed feedback on specifications of the force, instead of just being able to provide information that a force has been applied.

In an embodiment the external force is produced by; pelvic floor muscles, pelvic floor tissue and/or an abnormality such as an abscess present in the pelvic floor.

In an embodiment the external force is produced by a contraction of the levator ani.

In the following description the force applied by the levator ani muscles are used as an exemplary way of explaining the invention. The skilled person will recognize, that the invention will also be suitable for other pelvic floor muscles, tissues and/or an abscess.

The feedback from the apparatus may be in the form of a fail/success feedback, where success indicates that the levator ani was activated properly during the contraction and fail indicates that the levator ani was not activated or nor activated correctly during the contraction. In this way, the user will from the feedback provided from the apparatus be able to exercise the pelvic floor muscle more efficient and with higher quality, thereby reducing the time needed for strengthening the pelvic floor muscle and avoiding performing the exercise incorrectly.

In an embodiment the feedback is a visual feedback.

Advantageously, if the feedback is a visualisation of the contraction, the user can get a much more detailed feedback on how the pelvic muscle is activated during the contraction exercise. Hence, the user can thereby compare contractions, which enables muscle memory learning. Furthermore, as the apparatus is able to provide feedback to the user relating to the quality of pelvic floor exercises, the exercises can be done at home without the presence of a HPC, which saves both time, money, and is more convenient for the user

The apparatus of the present invention is a major advantage for HPC working within the field of treatment and/or strengthening of the pelvic floor muscle. The apparatus can in simple terms be understood as a digital finger that can collects novel, relevant and quantifiable user data. Hence, the need for palpation can be minimized and in some cases avoided. The data can be stored, graphed, tracked and used for research or for optimising patient care.

Advantageously, the data can be visualised to provide muscle profiles so as the HPC can better determine the present state of a pelvic floor muscle without having to perform a palpation. Thus, the apparatus provides the possibility for a HPC to examine the user without being in the same room both in real time or from stored data.

In terms of the present disclosure the term “sensor unit” can extend over the whole surface area of the sensor array, and can comprise one point of measurement or comprise of a plurality points of measurements making up the sensor unit for detecting a position of the exterior force applied to the outer surface in relation to the surface area of the surface array.

The terms “points of measurement” and “measurement points” are used interchangeable in the present disclosure.

In terms of the present disclosure the term “sensor array” is to be understood as an array which provides power and/or communication between the sensor unit and the micro-control unit.

The term relative is meant to clarify that the position, duration and magnitude of force applied from the contraction of a user is measured in relation to the force applied from the surface of the body on the sensor unit.

In terms of the present invention the term “magnitude” is to be understood relative size of the force applied to the body from a user.

The sensor unit may be a versatile sensor unit being able to detect and measure multiple factors including the duration of a contraction.

In an embodiment the sensor unit is adapted for detecting a pressure profile of the exterior force applied to the outer surface of the body.

This configuration enables the pressure to be divided into small bits of information. This information can give a very accurate “picture” or profile of the muscles in use, thereby enabling a novel visualization.

A muscles pressure profile, is important in diagnosing and treating pelvic floor muscle related issues, as it give a unique possibility to determine said muscles condition. A pressure profile indicates the quality of a muscles contraction, the muscle cell density and size. The invention can thus give never before seen data and novel visualizations of the progression and condition of the pelvic floor muscles.

In an embodiment the sensor unit comprises at least 5 measurement points per cm.

This enables the apparatus to be effective in measuring the shape, size, condition and related data of said muscle.

In an embodiment the sensor unit measures the magnitude of the exterior force applied to the body.

By providing a sensor unit, which detects the relative magnitude exerted on the outer surface, the feedback from the invention to the user or HPC is further improved.

By measuring both the magnitude and the position it is possible to determine a muscle strength profile of the pelvic floor muscle.

As an example a muscle profile may be obtained for a woman in her early pregnancy. By the initial quantitative feedback provided from the apparatus, the HPC and the pregnant woman is able to set up goals for the woman for which strength and/or duration of contractions to achieve post pregnancy.

In a further embodiment the sensor unit comprises two point of measurements placed at a maximum distance to each other of less than 0.3 cm, preferably less than 0.2 cm, more preferred less than 0.1 cm.

In an embodiment the distance between the two points of measurements is approximately 0.05 cm.

The resolution and level of detail of the feedback such as muscle profiles are enhanced, when arranging point of measurement closer to each other. In this way, the HPC get much more precise, accurate, quantitative data from the user. The visual muscle profiles obtained from the apparatus of the invention can result in quicker and more accurate diagnose. Thus, providing the most optimal treatment and/or training exercises for the user going forward.

As each point is arranged closer together the resolution of the visual muscle profiles increases.

In an embodiment the sensor unit is an optical sensor, preferably a force sensor, more preferably a force-resistive sensor.

According to the invention the micro-control unit can in an embodiment perform data analysis so as to provide feedback to the user via an indication on the apparatus or via communication through an external device.

In terms of the present disclosure, the term point of measurement can be defined in different terms dependent on the type of sensor. However, the skilled person will be able to determine the number of point of measurements for all types of sensors.

As an example, in the case of force-resistive sensor one point of measurement may be defined as a point having two electric contact plates separated by a small distance adapted to measure the change in resistance when pressure is applied between the two contact plates. Hence in an embodiment, the point of measurement is a single sensor cell, preferably a force sensing resistor.

In an embodiment the sensor unit comprises at least 10 measurement points, preferably at least 26 measurement points, more preferred at least 50 measurement points.

In an embodiment the sensor unit comprises between 52 to 104 measurement points.

In an embodiment, the sensor unit measures the relative distributed force applied to said sensor unit from a muscle contraction.

In terms of this disclosure the “distributed force” may be understood as normalized data for comparison between different users, there the magnitude of the force applied to each measurement point is calculated on the basis of the point receiving the largest force. However, the skilled person will know other ways of normalizing data.

In another embodiment, the sensor unit measures the relative distributed force applied to the point of measurement from a muscle contraction.

For providing superior feedback to the user, the visualization can be made from a relative distribution of the exterior force applied to a number of measurement points. As the pelvic floor muscle can have different characteristic at least in regard to strength, endurance of contractions and the shape of the muscle, the inventor has found that the optimal way of identifying the use of a muscle during a contraction is to analyze the distribution of the total applied force over a number of measurement points. Hence, the distribution of force (also referred to as the profile) may be used as a “finger print” for determining a certain muscle, tissue and/or abscess.

In a further embodiment of the invention, the sensor unit is a force sensor, preferable a force-resistive sensor.

In an embodiment the sensor array extends substantially along the whole length of the body, where the length of the body is defined as extending from the distal end to the opposite, proximal end.

In an embodiment the sensor unit or sensor units extends substantially along the whole length of the body.

In an embodiment the sensor unit is adapted to detect a position of a force applied anywhere on approximately the entire outer surface of the body.

This may be referred to as being able to measure an applied force in a 360 degree manner.

The 360 degree manner is to be understood as the sensor units being positioned so that regardless of where on the surface of the body a force is applied, the sensor units are capable of measuring in the entire circumference of the body.

It is understood that one sensor unit may be arranged so as to measure a force applied in an area covering a 0-90 degree angle of a radial direction of the body, where another sensor unit is arranged so as to measure a force applied in an area covering a 90-180 degree angle of the radial direction of the body, yet another sensor unit is arranged so as to measure a force applied in an area covering a 180-270 degree angle of the radial direction of the body, and another sensor unit is arranged so as to measure a force applied in an area covering a 270-360 degree angle of the radial direction of the body, so that the sensor units combined will detect a force applied anywhere on the circumference of the body.

In the terms of the present disclosure the term “circumference” is to be understood as the enclosing boundary of a geometric figure. The geometric figure may be a square, super ellipsis, rectangle, circle and any other geometric shape suitable for the body and/or a support structure (as explained below).

When the contraction from the user is measured in this way, it is possible to make a 2D and/or 3D-visualisation of the muscle contraction.

In a further embodiment the body comprises a sensor support structure, the sensor array being mounted on said support structure.

It has been found that the easiest and most cost efficient way to produce the body is by fitting the sensor array to a support structure. Furthermore, the support structure allows the stress and strain on the sensor array to be reduced and thereby prolonging the lifespan of the apparatus significantly.

For further reducing the stress and strain on the sensor array, in an embodiment the support structure comprises a flattened portion on to which the sensor array may be fitted.

By mounting the sensor array on a substantially flat surface, the strain within the sensor array and sensor unit is remarkably reduced. Hence, the lifetime of the apparatus and the accuracy and/or precision of the exterior force measurement can be increased.

In an embodiment of the invention, the support structure is fitted with bilateral arranged sensor arrays.

In an embodiment of the invention, the support structure is substantially rectangular.

In an embodiment of the invention, the apparatus comprises two sensor arrays arranged along the length of apparatus at an angle of approximately 85-95 degrees to each other.

In an embodiment of the invention, the apparatus comprises at least three sensor arrays arranged along the length of apparatus at an angle of approximately 85-95 degrees to the adjacent sensor array.

In an embodiment of the invention, the body comprise a top sensor arranged in the distal end of the body.

A top sensor can measure the presence of applied force from the abdomen.

Force from the abdomen is not desirable when exercising the pelvic floor muscle and this feedback is therefore a valuable feedback for the user and the HPC.

In another embodiment of the invention the apparatus comprises a communication module for communicating the data from the sensor array via the micro-control unit to an indicator and/or an external device.

The external device may be a computer means. The computer means may comprise visualization means and/or display means.

In yet another embodiment, the communication between the apparatus and the external device is a two-way communication.

In this way the feedback from the apparatus can be send to external device or similar, and the external device can give information to the apparatus.

In an embodiment the feedback from the apparatus is used for monitoring muscle contractions for profiling orgasms.

In an embodiment of the invention the body is made from silicone, thermoplastic, injection-moulded ABS plastic, and/or any milled material.

In another embodiment, the body is further coated with a relative soft material selected from the group of; medical silicone, latex, electroactive polymer and/or nitrile.

In yet another embodiment the body has a super elliptical shape, ellipsoid, blimp-like shape or torpedo-like shape.

The inventor has found that super elliptical shape of the body is preferred since the shape makes it comfortably for a user to insert the body into an opening and at the same is the optimal shape for getting accurate and precise contraction data.

In an embodiment of the invention, the apparatus comprises a head linked to the body.

The head may be linked to the body via a tail.

The head is used for encompassing electronical units, which does not function properly when inserted into a body opening. Hence, in a preferred embodiment, the communication module is encompassed in the head.

The head also provides additional safety in the form of an anchor that ensures, that the entire apparatus is not pushed all the way into the body opening. The head provides for easy grip and easy removal of the apparatus by the user.

Often signals from wireless communication such as Bluetooth or WI-FI are shielded when originating from a body opening, and typically it is preferred to have the wireless commutation arranged outside of the body opening.

In yet an embodiment, the apparatus comprises a tail for linking the body and the head both structurally and electronically.

The separation of the body and head is preferred for the user, since it enables the body to be made in smaller size (since it has to contain less elements) thereby providing for a better user experience.

In an embodiment of the invention, the tail is made from a flexible material.

The flexible tail provides the user with a higher degree of freedom for placing the head in a position that does not adversely affect the pelvic floor muscle exercise.

In an embodiment when the apparatus is inserted into a body cavity, the external force is applied by a pelvic floor muscle.

In an embodiment when the apparatus is inserted into a body cavity, the external force is applied by a levator ani muscle.

In an embodiment the apparatus further comprises a visualizing means for visualising the position of the applied external force and/or the magnitude of the external force and/or other relevant data.

In yet another embodiment, the pelvic floor muscle training and profiling apparatus is used for visualizing the movement of levator ani.

In an embodiment of the invention the pelvic floor muscle training and profiling apparatus can be used for visualizing the pelvic floor contraction of a user.

In another embodiment the visualisation of the contraction is displayed in a single dimension, double dimension, third dimension and/or multiple dimensions.

In difference to the prior art, the pelvic floor muscle contraction data can be used for visualising the movement and/or condition and/or state of the pelvic floor muscle, which can be a supplement or used instead of palpation performed by a HPC.

In an embodiment the invention relates to a use of pelvic floor muscle contraction data of a user for visualizing the movement of a pelvic floor muscle.

In an embodiment the invention relates to a use of pelvic floor muscle contraction data of a user for visualizing the movement of levator ani.

By visualizing the movement of levator ani, the user may be provided with a fail/success based feedback, which improves the quality of the pelvic floor muscle exercises.

According to a second aspect, the invention relates to a method for visualizing pelvic floor muscle contractions, comprising the steps of:

a) providing an apparatus comprising;
a body having a distal end, an opposite proximal end and an outer surface, the body extending between said ends,
a sensor array encompassed in the body, the sensor array comprising a sensor unit, the sensor unit being adapted for detecting an exterior force applied to the outer surface of the body,
a micro-control unit adapted to receive sensor data from the sensor array,
a visualising means adapted for receiving sensor data from said micro-control unit,

b) inserting said apparatus into a body cavity of a user,

c) the user contracting at least one pelvic floor muscle,

d) measuring the contraction on the outer surface of the body,

e) displaying the data of the pelvic floor muscle contraction on the visualising means.

This method ensures that the sensor data can be obtained with high precision and accuracy, while at the same time providing an apparatus which is simple in both structure and use, resulting in a cost efficient production and a superior user experience.

In an embodiment the data of the pelvic floor muscle contraction comprises; the position of the contraction, and/or the magnitude/size of the contraction and/or the movement of the muscle and/or the condition of the muscle and/or the relative distributed force and/or the muscle fingerprint.

Where the muscle fingerprint is defined as the profile of the muscle's impact on the apparatus.

In an embodiment the visualisation means is adapted to provide the user with feedback on the correctness of the muscle contraction.

In the terms of the present disclosure the term “correctness of the muscle contraction” is to be understood as the invention being able to compare a contraction profile (the profile may be normalized) of the external force applied to the apparatus, with a standard profile (which also may be normalized), so that the invention can inform the user, if the contraction is done correctly or if not, then how to improve the contraction so that it may be done more correctly.

In an embodiment of the invention is provided a method for visualizing pelvic floor muscle contractions, comprising the steps of:

a) inserting an apparatus of claim 1 into a body cavity of a user,

b) the user contracting pelvic floor muscles,

c) measuring the contraction on the outer surface of the body relative to the surface area of the sensor array,

d) displaying the data of the contraction on a display screen for visualising the pelvic floor muscle contraction.

In an embodiment the invention is provided a method for producing a pelvic floor muscle training and profiling apparatus comprising the steps of:

a) providing a sensor support having a distal end, an opposite proximal end and a support surface, the sensor support extending between said ends,

b) providing a sensor array comprising a sensor unit, which is adapted for detecting the position of an exterior force applied to the sensor unit in relation to the surface area of the sensor array.

c) attaching the sensor array to the sensor support structure

d) providing electronical communication between a microchip for receiving sensor data from the sensor array and the sensor array to provide a sensor system

e) encompassing the sensor system in an additional layer so as to provide the apparatus with final structure.

In another aspect the invention relates to a use of a pelvic floor muscle training and profiling apparatus according to the first aspect for visualizing the pelvic floor muscle contraction of a user.

In another aspect the invention relates to a use of a pelvic floor muscle training and profiling apparatus according to the first aspect in a method of visualizing the pelvic floor muscle contraction of a user according to the second aspect.

In an embodiment the invention relates to a use of pelvic floor muscle contraction data of a user for visualizing the movement of a pelvic floor muscle.

In an embodiment the invention relates to a use of pelvic floor muscle contraction data of a user for visualizing the movement of a levator ani muscle.

In another aspect the invention relates to a method for producing a pelvic floor muscle training and profiling apparatus comprising the steps of:

a) providing a sensor support structure having a distal end, an opposite proximal end, the sensor support structure extending between said ends,

b) providing a micro-control unit and a sensor array comprising a sensor unit, said sensor array is adapted for detecting the position of an exterior force applied to the outer surface of the body

c) attaching the sensor array to the sensor support structure

d) providing power and communication lines between the micro-control unit for receiving sensor data and the sensor array to provide a sensor element.

e) encompassing the sensor element in an additional layer so as to provide the apparatus with a final structure.

The sensor element comprises; the sensor support structure, the sensor array and the micro-control unit.

In a further embodiment the sensor array is attached as a sensor leaf comprising a sensor array, a sensor unit, a sensor resistive material and adhesion means for holding the leaf in place and for attaching the leaf to the sensor support structure.

In yet another embodiment the shape of the sensor support structure is an octagon.

Additional objects and advantages of the embodiments will be set forth in part in the description that follows, and in part will be obvious from the description or may be learned by practice of the embodiments. The objects and advantages of the embodiments will be realized and attained by means of the elements and combinations particularly pointed out in the appended claims.

It is to be understood that both the foregoing general description and the following detailed description are exemplary and explanatory only and are not restrictive of the claims.

The invention will now be described in greater detail. Each specific embodiment and variation of features applies equally to first aspect and the following aspect of the invention unless specifically stated otherwise.

### BRIEF DESCRIPTION OF THE DRAWINGS

The accompanying drawings, which are incorporated in and constitute a part of this specification, illustrate the disclosed embodiments and, together with the description, serve to explain the principles of the disclosed embodiments. In the drawings:

FIG. 1 illustrates the female anatomy of the lower abdomen;

FIG. 2 illustrates an exemplary pelvic floor muscle training and profiling apparatus, according to an embodiment of the disclosure;

FIG. 3 illustrates a side-view of an exemplary pelvic floor muscle training and profiling apparatus, according to an embodiment of the disclosure;

FIG. 4 illustrates an exemplary pelvic floor muscle training and profiling apparatus, according to an embodiment of the disclosure;

FIG. 5A illustrates an exemplary pelvic floor muscle training and profiling apparatus seated in an exemplary housing unit, according to an embodiment of the disclosure;

FIG. 5B illustrates the exterior of an exemplary housing unit in the closed position, according to an embodiment of the disclosure;

FIGS. 6A and 6B illustrates exemplary schematic diagrams of an exemplary pelvic floor muscle training and profiling apparatus according to embodiments of the disclosure;

FIG. 7A illustrates an exploded view of an exemplary sensor array for use in a pelvic floor muscle training and profiling apparatus, according to an embodiment of the disclosure;

FIG. 7B illustrates an assembled view of an exemplary sensor array for use in a pelvic floor muscle training and profiling apparatus, according to an embodiment of the disclosure;

FIG. 7C illustrates the relative position of an exemplary sensor array inside of the body of a pelvic floor muscle training and profiling apparatus, according to an embodiment of the disclosure;

FIG. 7D illustrates the relative position of an exemplary sensor array inside of the body of a pelvic floor muscle training and profiling apparatus, according to an embodiment of the disclosure;

FIG. 7E illustrates the relative position of an exemplary sensor array inside of the body, the body being shown as a cross sectional view, according to an embodiment of the disclosure;

FIG. 8A illustrates exemplary data corresponding to non-contraction of the pelvic floor muscles while a person employs an exemplary pelvic floor muscle training and profiling apparatus, according to an embodiment of the disclosure;

FIG. 8B illustrates exemplary data corresponding to incorrectly performed Kegel exercises while a person employs an exemplary pelvic floor muscle training and profiling apparatus, according to an embodiment of the disclosure;

FIG. 8C illustrates exemplary data corresponding to correctly performed Kegel exercises while a person employs an exemplary pelvic floor muscle training and profiling apparatus, according to an embodiment of the disclosure;

FIGS. 8D and 8E illustrates exemplary ways of visualizing an exterior force applied to the training and profiling apparatus;

FIG. 8F illustrates an exemplary way of visualizing the non-relative force distributed with a sensor array;

FIG. 8G illustrates an exemplary way of visualizing the relative force distributed with a sensor array;

FIG. 9A illustrates a top-view of an exemplary visualization display;

FIG. 9B illustrates a side-view of an exemplary visualization display;

FIG. 10A illustrates an exemplary visualization of a correct external force from the levator ani;

FIG. 10B illustrates an exemplary visualization of an irregular external force from the levator ani;

FIG. 10C illustrates an exemplary visualization of a general pressure not suitable for training;

FIG. 11 shows a flow chart of an exemplary method for visualizing pelvic floor muscle contractions according an aspect of the invention;

FIG. 12 shows a flow chart of an exemplary method for providing a pelvic floor muscle apparatus according to an embodiment of the invention.

### DETAILED DESCRIPTION

Reference will now be made in detail to the exemplary embodiments of the present disclosure described below and illustrated in the accompanying drawings. Wherever possible, the same reference numbers will be used throughout the drawings to refer to same or like parts.

For purposes of this disclosure, a “body cavity” may include a vagina or a rectum. The body cavity may be on a female or a male.

Prior to providing a detailed description, the following overview generally describes the contemplated embodiments. Pelvic floor muscle training and profiling apparatus 10 of the current disclosure is configured to be inserted into a body cavity, such as the vagina or rectum, such that body 11 is completely inserted into the body and tail 12 and head 13 remain outside of the body.

Pelvic floor muscle training and profiling apparatus 10 includes a body 11. Body 11 is configured to be inserted into a body cavity, such as the vagina or rectum, with ease, facilitated by its streamline profile and soft material coating. Body 11 may be of several different sizes to accommodate variations in anatomy from person to person. Body 11 may envelope a sensor array 40 and a wireless charging unit 30. Sensor array 40 may be used to detect pelvic floor muscle contractions. Wireless charging unit 30 may be used, in conjunction with a housing unit 20, to charge the battery that powers pelvic floor muscle training and profiling apparatus 10.

Connected to body 11 of pelvic floor muscle training and profiling apparatus 10 is a tail 12. The outer material of tail 12 encases wires 35 linking sensor array 40 and wireless charging unit 30 to the electronic components housed in a head 13. Head 13 may house several electronic components including, but not limited to, indicator lights 31, a battery 32, a communication module 33, and a micro-control unit 34.

When pelvic floor muscle training and profiling apparatus 10 is employed, body 11 is inserted into the body cavity, whereas tail 12 and head 13 remain outside of the body cavity. Sensor array 30 detects a user's muscle contractions and provides feedback to the user and/or the user's HPC. This feedback may be provided via indicator lights 31 on head 13 or by wireless communication (via communication module 33) to and displayed on a device such as a phone, a tablet, or a computer.

When pelvic floor muscle training and profiling apparatus 10 is not in use, it may be stored in housing unit 20. While stored in housing unit 20, pelvic floor muscle training and profiling apparatus 10 may be wirelessly charged, with housing unit 20 wirelessly providing electrical current to wireless charging unit 30 of pelvic floor muscle training and profiling apparatus 10. Pelvic floor muscle training and profiling apparatus 10 may indicate its battery charging status via the indicator lights 31 on head 13.

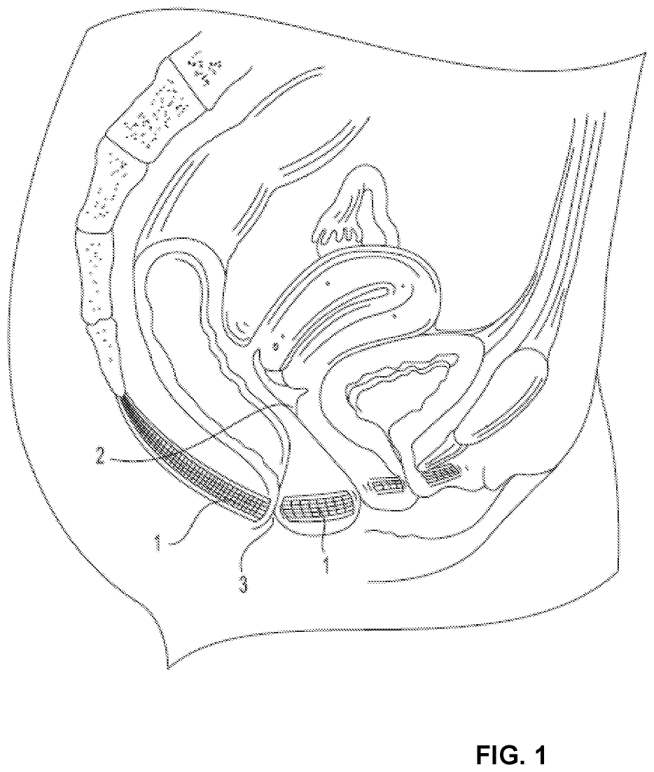

Reference is now made to FIG. 1, which illustrates the female anatomy of the lower abdomen. Body 11 of pelvic floor muscle training and profiling apparatus 10 may be inserted into either a vagina 2 or a rectum 3. While body 11 is inserted into either body cavity, contraction of pelvic floor muscles 1 may be measured. Especially, the movement of Levator ani is of the essence.

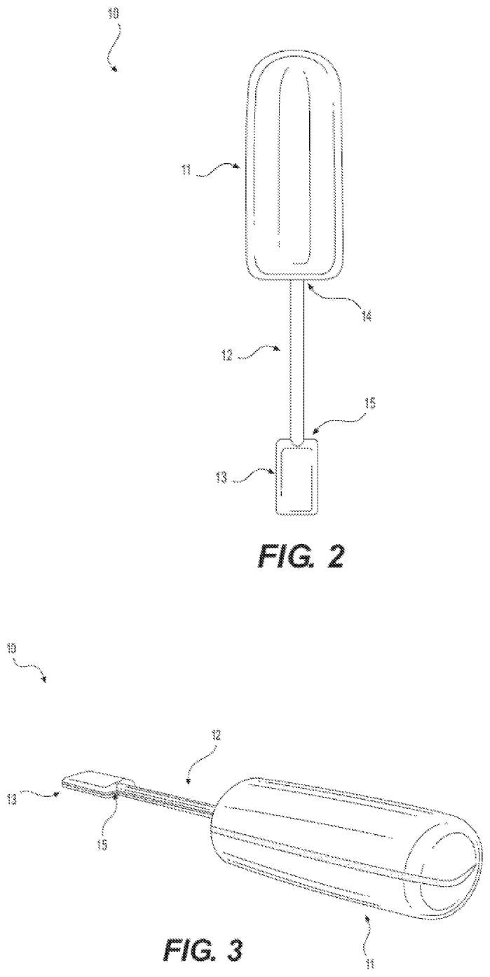

Reference is now made to FIG. 2, which illustrates an exemplary pelvic floor muscle training and profiling apparatus 10. As previously discussed, pelvic floor muscle training and profiling apparatus 10 may include body 11, tail 12, and head 13. Body 11 may be suitably shaped for vaginal or rectal insertion. In an exemplary embodiment, body 11 can have a super elliptical shape, possessing a flattened portion on to which the sensor or sensors can be fitted to help obtain uniform deformation data. Further, illustrative embodiments are of a super elliptical outer body shape, with or without this shape implemented internally. The shape of the super elliptical outer body can be defined by the parametric equations:

$$\begin{matrix}a \frac{cos ( t )}{\sqrt{cos ( {t}^{2} )}} \cdot {( \sqrt{cos ( {t}^{2} )} )}^{2 / N} & Eq . 1 \\ b \frac{sin ( t )}{sin ( {t}^{2} )} \cdot {( \sqrt{sin ( {t}^{2} )} )}^{2 / N} & Eq . 2\end{matrix}$$

where a and b can vary, and N ranges from 1 to 7.

Additional suitable shapes for body 11 may in other illustrative embodiments include an ellipsoid, blimp-like shape or torpedo-like shape, as illustrated in FIG. 1. The rounded, distal (opposite side of tail attachment) end of body 11 may facilitate a pain-free insertion into the body cavity. Body 11 may house sensor unit 30. Additional components such as a wireless charging unit can also be provided. Body 11 may be made of a relatively hard material, such as, for example, thermoplastic, injection-molded ABS plastic, or any milled material, and may be coated with a relatively softer material, such as, for example, medical silicone, latex, electroactive polymer or nitrile.

An optimal outer coating for body 11 is necessary so that when body 11 is inserted into a body cavity, contractions by pelvic floor muscles 1 may be measured by sensor unit 30. Parameters such as hardness, elongation at break, tensile strength, tear strength, Young's modulus, and linear shrinkage may be optimized to provide a coating that can effectively measure muscle contractions. For example, in one embodiment, the Shore A hardness may be about 22°, the elongation at break may be about 497%, the tensile strength may be about 3.64 MPa, the tear strength may be about 26.24 kN/m, the Young's modulus may be about 1.5 MPa, and the linear shrinkage may be about 0.4%.

Tail 12 is designed to link body 11 to head 13 both structurally and electronically, and may be flexible. At body/tail mount point 14 and head/tail mount point 15, strain relieves may be employed to ensure that bending, during use and storage, does not damage the structure or internal components. The coating of tail 12, to be discussed further below, may be silicone, and will encase power and communication lines connecting the components in body 11 and head 13. The flexible nature of tail 12 may allow for a user to adjust tail 12 and head 13 to a comfortable position while using pelvic floor muscle training and profiling apparatus 10.

Head 13, which is attached to tail 12 opposite the end where body 11 is attached, remains outside of the body cavity during use. As previously discussed, head 13 may contain battery 32, communication module 33, micro-control unit 34, and indicator lights 31. Like body 11, head 13 may be made of a relatively hard material, such as, for example, thermoplastic, injection-molded ABS plastic, or any milled material, and may be coated with a relatively softer material, such as, for example, medical silicone, latex, electroactive polymer or nitrile. As illustrated in FIG. 2, head 13 may have a rectangular shape with rounded edges, or it may possess a shape such as, for example, super elliptical, round, or elliptical.

As previously discussed, pelvic floor muscle training and profiling apparatus 10 may be available in an array of sizes to accommodate a range of body sizes. Accordingly, body 11 may have a diameter ranging from about 15 mm to about 35 mm, and a length ranging from about 70 mm to about 90 mm. Tail 12 may have an outer diameter ranging from about 10 mm to about 60 mm, and a length ranging from about 70 mm to about 130 mm. And head 13 may have a length ranging from about 30 mm to about 100 mm, a width ranging from about 10 mm to about 50 mm, and a height ranging from about 10 mm to about 50 mm.

In one embodiment, body 11 of a small-sized pelvic floor muscle training and profiling apparatus 10 may have a diameter ranging from about 10 mm to about 25 mm, and a length ranging from about 50 mm to about 70 mm. In another embodiment, body 11 of an intermediate-sized pelvic floor muscle training and profiling apparatus 10 may have a diameter ranging from about 25 mm to about 35 mm, and a length ranging from about 60 mm to about 90 mm. In a further embodiment, body 11 of a large-sized pelvic floor muscle training and profiling apparatus may have a diameter ranging from about 35 mm to about 70 mm, and a length ranging from about 70 mm to about 150 mm.

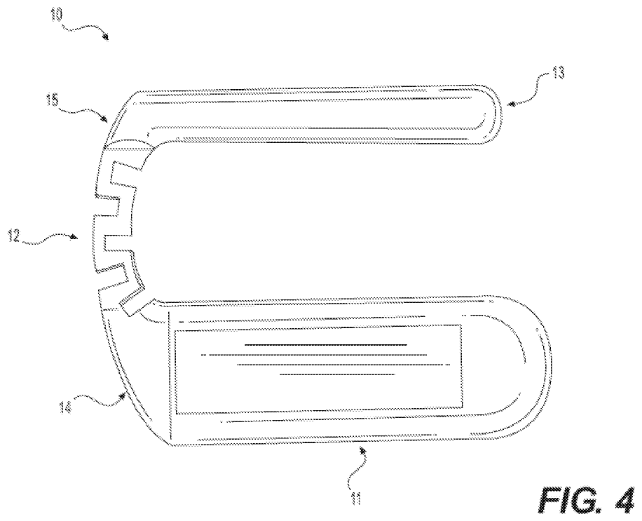

Reference is now made to FIG. 3, which illustrates a side-view of an exemplary pelvic floor muscle training and profiling apparatus 10, in accordance with an embodiment of the disclosure. Here, the streamline profile of body 11 can be seen, which may facilitate easy insertion into a body cavity.

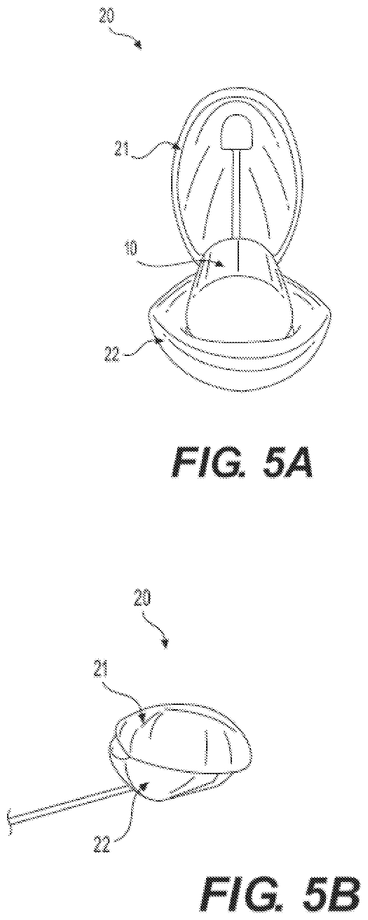

Reference is now made to FIG. 4, which illustrates an exemplary pelvic floor muscle apparatus 10, in accordance with an embodiment of the disclosure. The angles of body/tail mount 14 and head/tail mount 15, in conjunction with a relatively stiffer tail 12, give rise to a pelvic floor muscle apparatus 10 that possesses a U-shape. This U-shape may provide better body 11 positioning in the body cavity by stabilization via contact between head 13 and, for example, a person's labia. As illustrated in FIG. 4, tail 12 may have a tooth-like pattern; alternatively, tail 12 may have, for example, a curved or curly shape. In one embodiment, the longitudinal axis through the center of tail 13 may be about parallel to the longitudinal axis through the center of body 11. In another embodiment, the longitudinal axis through the center of head 13 may be from about 45° to about 90° offset from the longitudinal axis through the center of body 11. This offset configuration may avoid sexual stimulation by preventing contact between head 13 and the user's clitoris.

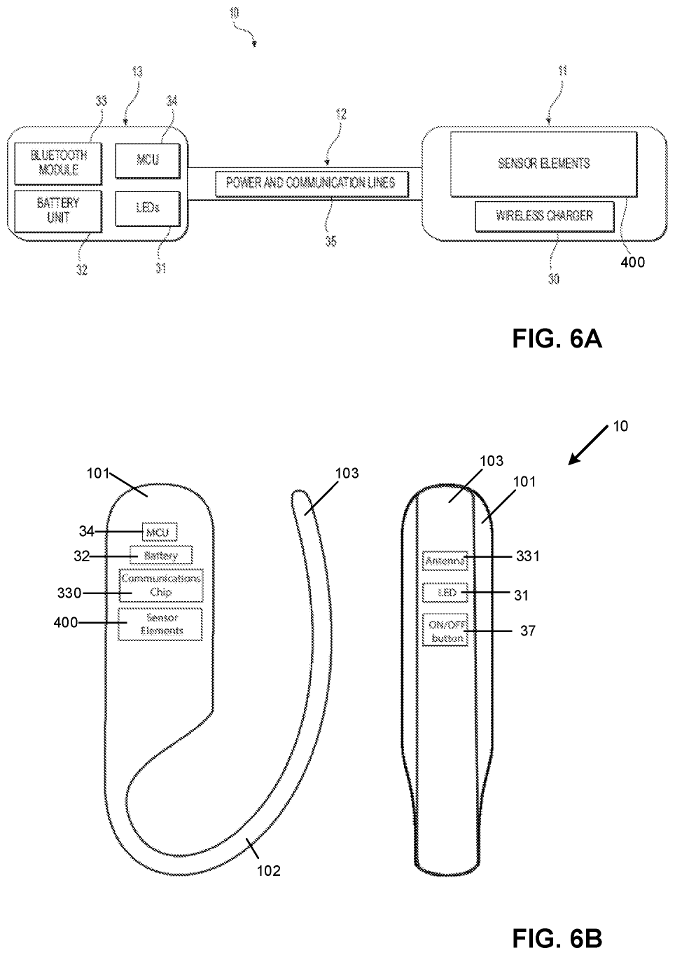

Reference is now made to FIG. 5A, which illustrates an exemplary pelvic floor muscle training and profiling apparatus 10 seated in an exemplary housing unit 20, in accordance with an embodiment of the disclosure. Housing unit 20 may serve as a charging unit for pelvic floor muscle training and profiling apparatus 10. When pelvic floor muscle training and profiling apparatus 10 is seated in housing unit 20, housing unit 20 may inductively charge pelvic floor muscle training and profiling apparatus 10 via wireless charging unit 30. Further, housing unit 20 may serve as a protective case for pelvic floor muscle training and profiling apparatus 10. While charging, pelvic floor muscle training and profiling apparatus 10 may emit light via indicator lights 31, located on head 13, to indicate the battery charging status. Housing unit 20 may be transparent or translucent in portions of a cover 21 or a base 22, and thus may allow the indicator light to be seen from the outside. Further, housing unit 20 may serve as a calibration tool for pelvic floor muscle training and profiling apparatus 10. For example, cover 21 may be a defined weight, and the force that it applies to pelvic floor muscle training and profiling apparatus 10 when closed can be used to calibrate sensor array 40.

Reference is now made to FIG. 5B, which illustrates an exemplary housing unit 20, in a closed position, in accordance with an embodiment of the disclosure. The space-saving nature of housing unit 20 is illustrated in FIG. 5B.

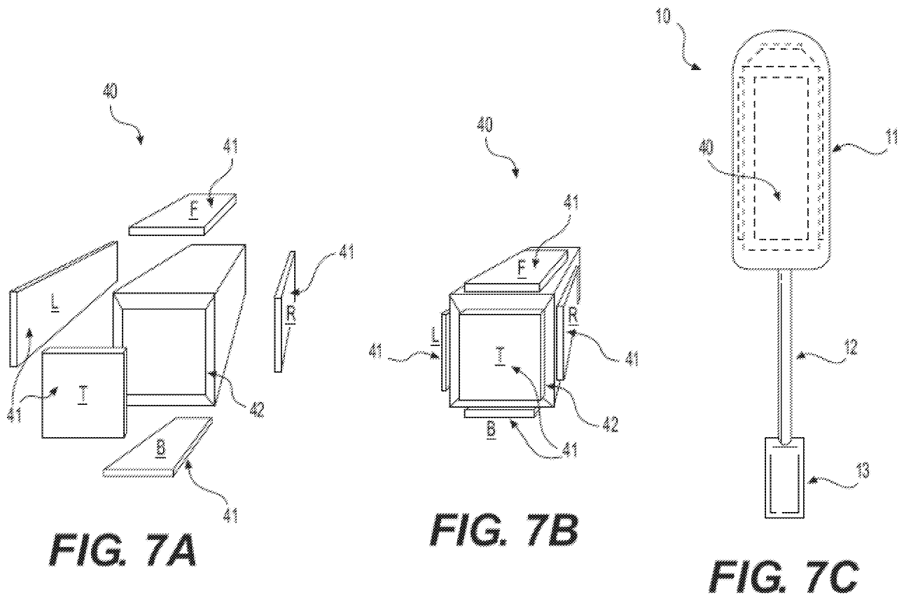

Reference is now made to FIG. 6A, which illustrates an exemplary schematic diagram of an exemplary pelvic floor muscle training and profiling apparatus 10, in accordance with an embodiment of the disclosure. Body 11 may encase sensor element 400 comprising a sensor support structure, sensor array, sensor unit described below, and wireless charger 30. As previously described, wireless charger 30 may wirelessly receive an electrical current from housing unit 20 to charge battery unit 32.

Wires 35, encased in tail 12, may facilitate power transfer and electronic communication between the components encased in body 11 and the components encased in head 13. There may be one or more wires 35 connecting the components in body 11 and head 13 via tail 12.

Head 13 may encase indicator lights 31, battery 32, communication module 33, and micro-control unit 34. Indicator lights 31 may provide visual feedback to a user concerning battery 32 charge state, connection state (for example, via communication module 33), and device power state (for example, on or off). In one embodiment, indicator lights 31 may be LEDs, or light emitting diodes. In another embodiment, indicator lights 31 may be fluorescent lights. Indicator lights 31 may provide visual feedback by, for example, blinking in pre-arranged patterns, varying brightness, maintaining a constant intensity, or by changing color. For example, in one embodiment, indicator lights 31 may emit a green light to indicate that battery 32 is fully charged.

Battery 32 is the power source for pelvic floor muscle training and profiling apparatus 10. It provides power to indicator lights 31, communication module 33, and micro-control unit 34, and sensor array 40 via wires 35. Further, in an illustrative embodiment, battery 32 is charged by wireless charging unit 30 via wires 35. In one embodiment, battery 32 may be a lithium ion battery. Any type of battery, rechargeable or non-rechargeable, can be used in various embodiments.

Communication module 33 serves to communicate data between pelvic floor muscle training and profiling apparatus 10 an external device, such as, for example, a smartphone, a tablet computer, a laptop, or a desktop computer. Communication between pelvic floor muscle training and profiling apparatus 10 and the external device via communication module 33 may be only a one-way transfer of information (from pelvic floor muscle training and profiling apparatus 10 to the external device), or it may be a two-way communication transfer. For example, in one embodiment, pelvic floor muscle training and profiling apparatus 10 may transmit muscle contraction data to an external device. In another embodiment, pelvic floor muscle training and profiling apparatus 10 may transmit operational status data, such as power state or battery charge status. Communication transfer is not limited to one external device; communication module 33 may transmit data to multiple devices. For example, in one embodiment, communication module 33 may transmit data, such as, for example, muscle contraction data, to an external device viewed by the user and to an external device viewed by a HPC.

Communication module 33 may communicate by one or more communication protocols. For example, in one embodiment, communication module 33 may transmit and receive data by Bluetooth communication. In another embodiment, communication module 33 may transmit and receive data via 802.11a/b/g/n communication. In another embodiment, communication module 33 may transmit and receive data by both Bluetooth and 802.11a/b/g/n communication. In a further embodiment, communication module transmits data via serial UART communication protocol.

Micro-control unit 34 is the central processing unit of pelvic floor muscle training and profiling apparatus 10. Micro-control unit 34 may receive muscle contraction data, as measured by sensor array 40, or it may receive the battery charging status, via battery 32 and wireless charging unit 30. Micro-control unit 34 may communicate this data to an external device via communication module 33. Further, micro-control unit 34 may communicate data, such as, for example, battery charge status, via indicator lights 31.

Reference is now made to FIG. 6B, which illustrate an embodiment of the apparatus 100 having a body 101, a tail 102 and a head 103. The head 103 having a similar shape as the tail 102.

The body 101 encompasses both the sensor elements 400, comprising a sensor array, sensor unit, sensor support structure and a communication chip 330 which is a part of the communication module. The antenna part 331 of the communication module is placed in the head 103, which in this embodiment is integrated with the tail 102. The head 103 further comprises an LED 31 and an on/off button 37.

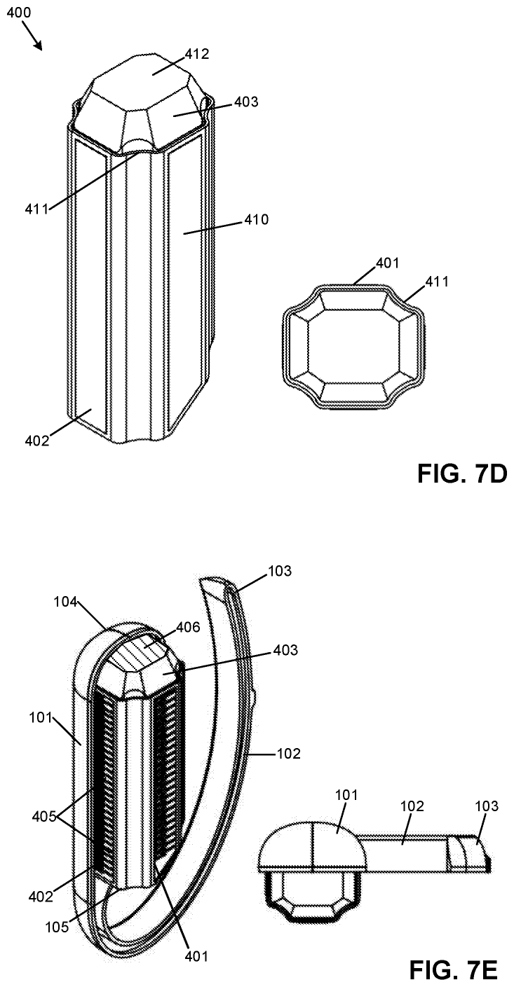

Reference is now made to FIG. 7A, which illustrates an exploded view of an exemplary sensor array 40 for use in a pelvic floor muscle training and profiling apparatus 10, in accordance with an embodiment of the disclosure. Sensor array 40 includes one or more sensor units 41 and a sensor support structure 42. Sensor units 41, when mounted on sensor support structure 42, work cooperatively to sense muscle contractions applied to the outside of body 11, in a 360° manner.

Sensor unit 41 in illustrative embodiments can include a pixelated deformation sensor system or a multilateral sensor array or both. Both a pixelated deformation sensor system and a multilateral sensor array may allow for the measurement of both the magnitude of the force applied and the position of the application of the force (along the length of body 11). Sensor unit 41 may be a sensor, or a method of sensing force, selected from a stiff mechanical transfer pressure sensitive or force resistive sensor; an optical sensor; a barometric sensor; a linear potentiometer sensor based upon mechanical displacement; a capacitive force sensor; straining gauges; optical frustration by internal reflection; optomechanical sensor; light coupling between adjacent fibers; optical tracking of markers in deformable gel; a combination of integral force measurement and positioning sensor; a surface electromyographic sensor; and an analog resistive matrix sensor. Each will be described in turn. In one embodiment, each sensor unit 41 may be the same type of sensor. In another embodiment, each sensor unit 41 may be a different type of sensor. A combination of different sensor types may provide a broader amount of data, as some sensors may be more sensitive than other sensors. In one embodiment, senor unit 41 may be a pressure profile sensor. In another embodiment, sensor unit 41 may be a combination of pressure profile sensor and S-EMG. In addition to sensing muscle contraction force, sensor unit 41 may be an accelerometer or a thermometer.

Sensor unit 41 may be a variety of dimensions and materials, depending upon, for example, the type of sensor(s) employed, the overall arrangement of sensors, and the size of body 11. In one embodiment, sensor unit 41 may have a length ranging from about 50 mm to about 60 mm, a width ranging from about 10 mm to about 15 mm, and a height ranging from about mm to about mm.

Sensor support structure 42 provides a structure for sensor units 41 to be mounted onto. Sensor support structure 42 may be fabricated from silicone, glass fiber, or plastic. The dimensions and shape for sensor support structure 42 may be a variety of dimensions and shapes, depending upon, for example, the type of sensor(s) employed, the overall arrangement of sensors, and the size of body 11.

Reference is now made to FIG. 7B, which illustrates an assembled view of an exemplary sensor array 40 for use in a pelvic floor muscle training and profiling apparatus 10, in accordance with an embodiment of the disclosure. As illustrated in FIG. 7B, many surfaces of support structure 42 have a sensor unit 41 mounted onto it; this configuration may provide for 360° sensing of pelvic floor muscle contraction.

Reference is now made to FIG. 7C, which illustrates the relative position of an exemplary sensor array 40 inside of a body 11 of a pelvic floor muscle training and profiling apparatus 10, in accordance with an embodiment of the disclosure. As FIG. 7C illustrates, sensor array 40 may extend for nearly the entire length of body 11. When configured as such, it may be possible to detect muscle contraction along the length of body 11 in a 360° fashion. In addition to 360° sensing, other amounts of sensing may be possible, for example, about 270°, about 180°, about 135°, and about 90° sensing.

Reference is now made to FIG. 7D, which illustrate a sensor element 400 according to the invention having a sensor support structure 403, which is essentially an octagon having 8 edges and 4 substantially flat sides 410. The rounded edges 411 of the sensor support structure 403 functions as a slidable surface for the pelvic floor muscle, so when a muscle is pressed towards the rounded edge 411 it will slide onto the sensor unit 402, where the position, magnitude and/or duration of the force is measured and then visualized.

The sensor unit 402 is a pixelated sensor unit having a plurality of point of measurements (not shown). On the top side 412 of the sensor support structure 403 may optionally be provided with a top sensor 412 for detecting a force applied from the abdominal.

Furthermore, the sensor array 401 is provided around the circumference of the sensor support structure 403 so as to detect the position of a muscle contraction in a 360 degree manner.

Reference is now made to FIG. 7E, which illustrates a perspective view of a cross section of the body 101 of the apparatus, the body 101 having the sensor support structure 403, the sensor array 401 and sensor units 402 arranged within. The senor array 401 is fitted to the sensor support structure 403 so as the sensor unit 402 is placed on the substantially flat sides.

In this particular embodiment the sensor unit 402 is a force sensing resistor. The measuring points 405 of the sensor unit 402 are arranged in four columns, wherein each column extends substantially the entire length of the body 101, said length is the distance from the distal end 104 to the proximal end 105.

Each point 405 in the sensor column is distanced with by 0.2 cm and the sensor array comprise a total of 100 measurement points 405 for obtaining a visualization with a good resolution.

Furthermore, the sensor unit 402 is provided around (substantially in the circumference) the sensor support structure 403 so as to detect the position of a muscle contraction in a 360 degree manner.

It is contemplated that the distance between the measurement points may be lower than 0.2 cm, and that the sensor type may vary independently of the number of measurement points.

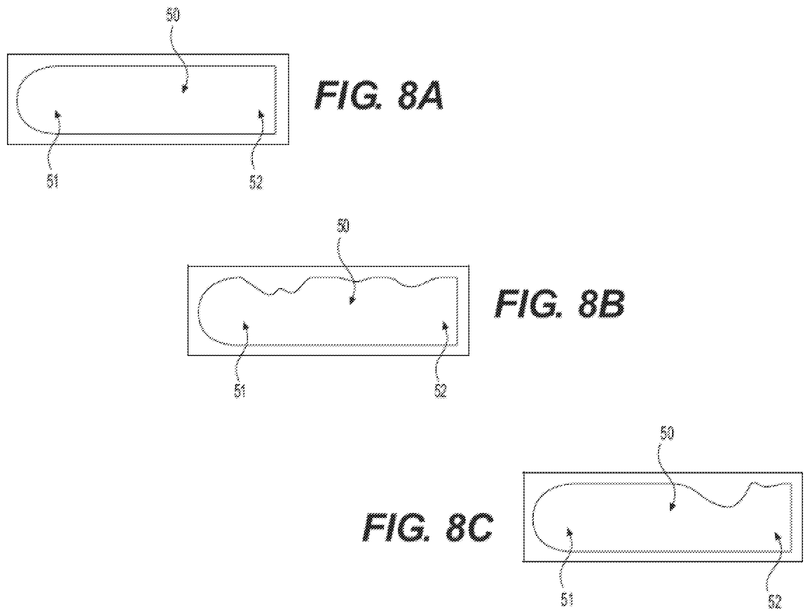

Reference is now made to FIG. 8A, which illustrates an exemplary feedback display 50, when pelvic floor muscles 1 are in a relaxed state, in accordance with an embodiment of the disclosure. Feedback display 50 illustrates a side-view of an exemplary body 11. When body 11 is inserted into a body cavity and the person is not contracting pelvic floor muscles 1, the internal sensors of body 11 will not detect any pressure (or a pressure beneath a threshold pressure), and thus not display a contraction.

Reference is now made to FIG. 8B, which illustrates an exemplary feedback display 50, when pelvic floor muscles 1 are in an improperly contracted state, in accordance with an embodiment of the disclosure. When body 11 is inserted into a body cavity and the person contracts pelvic floor muscles 1, the internal sensors of body 11 will detect the pressure exerted from the contracting muscles. This applied pressure may result in deformation of the soft coating of body 11, and the relative positioning of the force along the length of body 11, in addition to the relative magnitude of the contraction force, may be displayed as feedback display 50. When a patient improperly contracts pelvic floor muscles 1, other muscles may be engaged and thus multiple deformations may be detected, or a contraction too close to distal end 51 may be detected, as illustrated in FIG. 8B.

Reference is now made to FIG. 8C, which illustrates an exemplary feedback display 50, when pelvic floor muscles 1 are in a properly contracted state, in accordance with an embodiment of the disclosure. When body 11 is properly inserted into the body cavity, the deformation of body 11, resulting from muscle contraction, should occur near proximal end 52, or the end of body 11 closest to where tail 12 is attached, as illustrated in FIG. 8C.

FIG. 8D illustrates one exemplary way of visualizing an exterior force applied to the training and profiling apparatus;

FIG. 8E illustrates another exemplary way of visualizing an exterior force applied to the training and profiling apparatus;

FIG. 8F illustrates an exemplary way of visualizing the non-relative force distributed with a sensor array.

FIG. 8G illustrates an exemplary way of visualizing the relative force distributed with a sensor array.

Reference is not made to FIG. 8F which illustrates an exemplary way of visualizing the non-relative force distributed with a sensor array. In this embodiment feedback can be given to the user only on the basis of each point 405 measuring the position and force applied to that point. Hence, in this case it will be necessary to arranges the points 405 of measurement closer together such as to provide a higher resolution if for example the HPC is in doubt whether a peak originates from one or two muscles.

Reference is now made to FIG. 8G showing a preferred way of visualizing by measuring the relative force distributed between each measurement point 405. By having communication between each measurement point and the micro-control unit it is possible to provide a superior feedback by data analyses by means of algorithm.

The algorithm would search for the individual signals from the individual points and search for a profile (a number of active points) that match a pre-defined profile (e.g. a profile pertaining to the levator ani).

If levator ani is activated correctly, the user would be able to see and potentially hear by indication means such as speaker placed in the head that the clenching exercise is done correctly.

Thus the algorithm looks for the total number of sensor points and how the individual force impact is between them.

The data that is available to the HCP shows among other muscle size and condition, which allows the HCP to track muscle growth and density.

The profiling and the algorithm allows for an individual training program in which the apparatus informs the user when a correct clench is performed. This feedback will prevent the user form potentially making wrong exercises and thereby harming themselves or the training.

Moreover, not only the levator ani is of interest, but also all the other pelvic floor muscles. However, it is used as an example since it is the primary muscle in that area.

Besides the pelvic floor training and monitoring, the apparatus may also be used in a sexual application, wherein the apparatus may examine orgasms and general muscle contractions, thereby enabling the apparatus to be user specific (e.g. via machine learning), so that the apparatus may vibrate depending on whether an orgasm is coming or not. The visualized muscle profile includes much more information than just the size of the external force. The muscle profile is very important in order to understand the precise state or condition of the muscle.

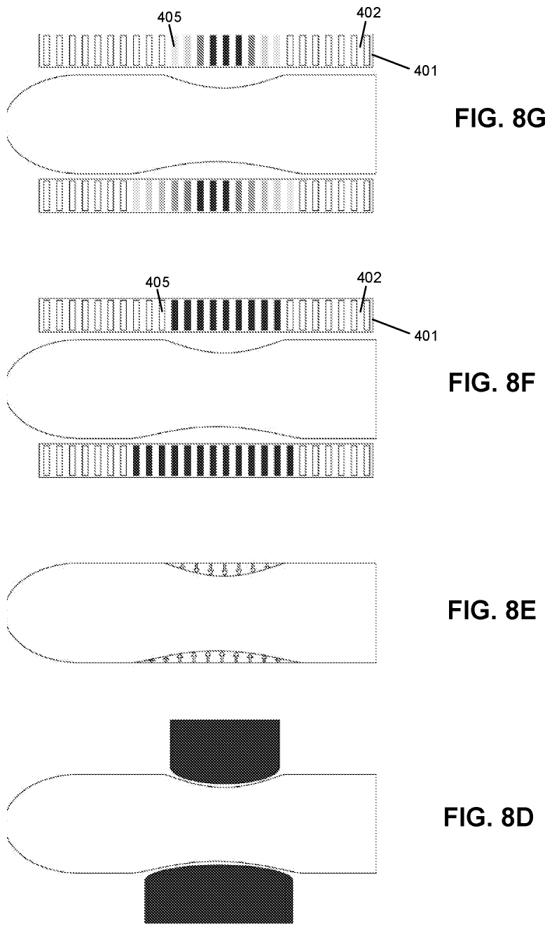

Reference is now made to FIGS. 9A and 9B, which illustrates an exemplary feedback display 40, in accordance with an embodiment of the disclosure. Feedback display 50′ illustrates a top-view in FIG. 9A and a side-view in FIG. 9B of an exemplary visualization display. When body 11 is inserted into a body cavity and the person is contracting pelvic floor muscles 1, the internal sensors of body 11 will detect pressure, and thus display a contraction. Other displays are of course possible.

As previously described, correct pelvic floor muscle contraction may lead to a strengthening of that muscle, which may assist with certain medical conditions. Pelvic floor muscle training and profiling apparatus 10 may be used in a body cavity, for example, a vagina or a rectum. A user may use the device alone, or in the presence of her or his HPC.

When pelvic floor muscle training and profiling apparatus 10 is employed, feedback may be provided to an external device. Examples of an external device may include smartphones, tablet computers, laptop computers, and desktop computers. Data displayed may be in the form of a diagram, for example, a diagram as illustrated in FIGS. 8A, 8B, and 8C. A HPC may view the data and provide feedback to the user to assist the user in proper contraction technique. The HPC may view data on apparatus 10 user's external device (using an accessory detachable mirror for convenience if desired), on the HPC's own external device receiving direct, wireless communication from the user's pelvic training and profiling apparatus 10, or via a remote connection if the user is not using pelvic floor training and profiling apparatus 10 in the presence of the HPC.

A user may be able to determine if pelvic floor muscle contractions are performed properly in the absence of physician feedback. For example, based upon the data display on an external device, a user may be able to determine if the contractions are performed properly, based upon experience or guidance from a HPC. Another example includes an application on the user's external device that evaluates the data and provides feedback, in a similar manner to feedback provided by a physician, as to the correctness of the muscle contraction.

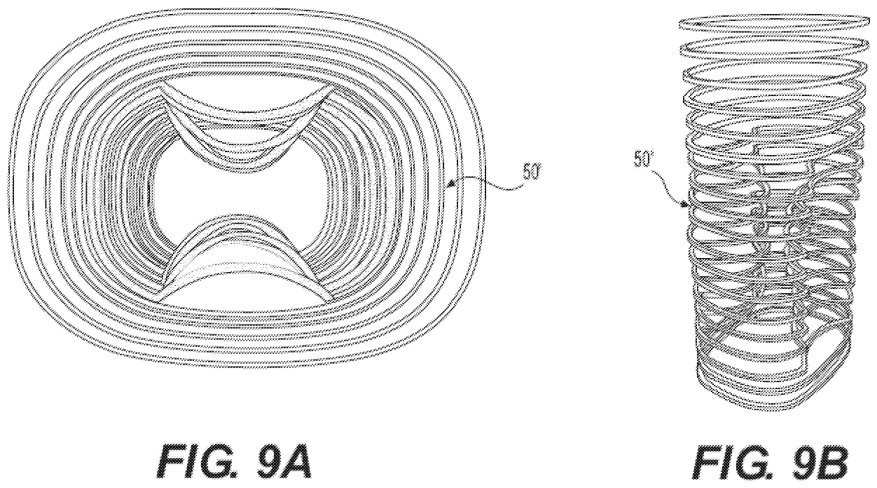

FIG. 10A illustrates an exemplary visualization of an regular external force applied by the levator ani.

FIG. 10B illustrates an exemplary visualization of an external force in form of a general pressure.

FIG. 10C illustrates an exemplary of an irregular external force applied by the levator ani.

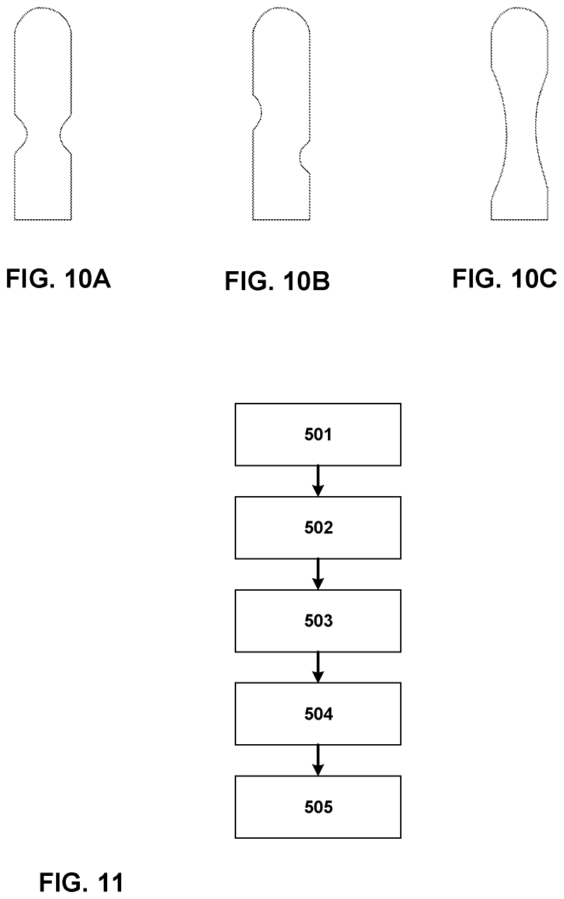

Referring to FIG. 11 which shows a flow chart of an exemplary method for visualizing pelvic floor muscle contractions according an aspect of the invention.

The flow chart illustrates the following steps of the method:

501) providing an apparatus comprising;
a body having a distal end, an opposite proximal end and an outer surface, the body extending between said ends,
a sensor array encompassed in the body, the sensor array comprising a sensor unit, the sensor unit being adapted for detecting an exterior force

applied to the outer surface of the body,
a micro-control unit adapted to receive sensor data from the sensor array,
a visualising means adapted for receiving sensor data from said micro-control unit,

502) inserting said apparatus into a body cavity of a user,

503) the user contracting at least one pelvic floor muscle,

504) measuring the contraction on the outer surface of the body,

505) displaying the data of the pelvic floor muscle contraction on the visualising means.

Hereby the user is able to receive a visual feedback regarding the muscle contractions. The feedback may include information regarding the performance of the muscle contractions, so that the user may improve said contractions.

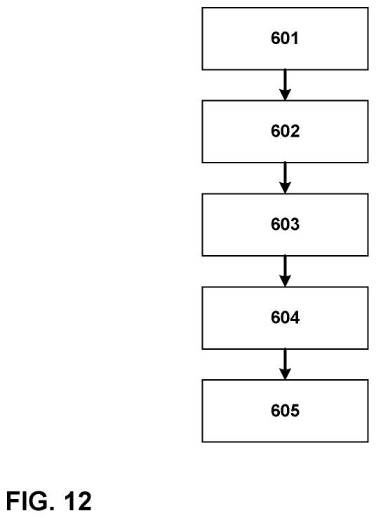

Referring to FIG. 12 which shows a flow chart of an exemplary method for providing a pelvic floor muscle apparatus according to an embodiment of the invention.

The method comprising the steps of:

601) providing a sensor support structure having a distal end, an opposite proximal end, the sensor support structure extending between said ends,

602) providing a micro-control unit and sensor array comprising a sensor unit, which is adapted for detecting the position of an exterior force applied to the sensor unit in relation to the surface area of the sensor array.

603) attaching the sensor array to the sensor support structure

604) providing power and communication lines between a micro-control unit for receiving sensor data and the sensor array to provide a sensor element.

605) encompassing the sensor element in an additional layer so as to provide the apparatus with final structure.

The sensor support structure can be manufactured by 3D-printing, injection moulding or similar techniques. In a further embodiment, the sensor support structure material is preferably a low density and rigid material so as to aid the weight minimization of the entire apparatus and still provide a mechanical stable structure.

In an embodiment the material of the sensor support structure is a thermoplastic, preferably polypropylene or acrylonitrile butadiene styrene such as ABS-M30i sold by the company Stratasys.

The shape of the sensor support structure is preferably an octagon with 4 rounded edges and 4 substantially flat portions, wherein the sensor unit is placed on the flat portions of the sensor support structure to reduce the strain on the sensor unit.

In a particular embodiment where the sensor unit is a force resistive sensor, the sensor array and sensor unit may advantageously be provided as a leaf sensor comprising a micro-control unit connected to the sensor array. The sensor array provides a scaffold for the sensor unit, and provides power and communication lines between the micro-control unit and the sensor unit.

In a further step of producing a leaf sensor, an additional layer of a force resistive material is mounted on the top side of the sensor unit connected to sensor array. The layer of the force resistive material may cover the whole surface area of the sensor array and is preferably provided in oversize.

In yet another step of providing a sensor leaf, a sticky layer is provided to bottom side of the sensor array and on the top side of the force resistive material to provide the finished leaf sensor. The sticky layers ensure that the sensor array, sensor unit and the force resistive material is held in place.

In an embodiment of the invention the sticky layer is double sided tape.

In a further embodiment, the leaf sensor can be attached to the sensor support structure by means of adhesion, preferably by double sided tape.

Advantageously, by attaching a sensor leaf to the sensor support structure, the step of encompassing the sensor element may be performed without any material penetrates the leaf and get into the electronic and sensor portion of the sensor leaf. Hence, in one embodiment the sensor leaf is impermeable to liquids.

The step of encompassing the sensor element is preferably done by moulding an additional layer of liquid silicone such as ELATOSIL® LR 3003 sold by Wacker Chemie onto the sensor element. Silicone renders the apparatus physically inert, inhibits the growth of bacteria, reduce the development of odours and enable easy cleaning of the apparatus. In an embodiment of the invention, the thickness of the layer encompassing the sensor element is a maximum of 2 mm to ensure the force applied from the contraction of a user is measured by the sensor unit.

Typically, the micro-control unit and sensor is connected in structure, power and communication lines before attaching the sensor array to the sensor support structure.

In a further embodiment the micro-control unit is linked to the tail.

The final structure may be any shape possible to mould, but preferably a super elliptical shape.

It is contemplated when the sensor unit is not a force resistive sensor, the step of providing a force resistive material may be omitted for producing a sensor leaf.

Muscle Profiling

In the following section different examples will be given on how to use the apparatus and the feedback derived from said apparatus.

In a first example the apparatus according to the invention may be used for training the pelvic floor muscle. By focusing on either the different specialized muscles of the pelvic floor or particular muscles solo or in group, the strength of the pelvic muscle can be trained by measuring from where the exterior force is applied on the outer surface, the magnitude of the force and the duration of the force applied. In any case, the feedback can be used to make an individual training plan in relation to the present state of the pelvic floor muscles and dependent on the goal of the user.

In a further example, the apparatus can be used for sexual pleasure. Hence, in an embodiment of the invention the apparatus is a sex toy, such as a dildo or a vibrator. As different orgasms and/or pre/post orgasm muscle movement/contractions may give different muscle profiles, the collection and process of data may give the user an enhanced information on how to increase the sexual pleasure during masturbation or intercourse.

In an embodiment of the invention the apparatus may comprise an actuator, wherein the actuator receives an input from the micro-control unit. The input may activate and/or deactivate the actuator. The actuator may in an active condition provide vibration and/or pulsating movement for the pleasure of the user.

In another example, the apparatus can be used for women suffering from prolapse. After childbirth many women are suffering from strained pelvic floor muscles, which leads to prolapse of i.e. bladder, uterus. In this case, a pressure profile can be used to strengthen the muscle in the lower belly, thus easing the prolapse, and perhaps avoiding surgery.

In a further example, the apparatus can be used for diagnosing and treating vaginism. A Muscle profile measured through insertion rectally may provide the health care professional with knowledge of which muscle that spasms and causes the condition.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description.

1. A pelvic floor muscle training and profiling apparatus for inserting into a body cavity of a human, said apparatus comprising:
a body having a distal end, an opposite proximal end and an outer surface, the body extending between said ends,
a sensor array encompassed in the body, the sensor array comprising a sensor unit, the sensor unit being adapted for detecting an exterior force applied to the outer surface of the body,
a micro-control unit adapted to receive sensor data from the sensor array, characterized in that the apparatus further comprises a top sensor arranged in the distal end of the body, and the sensor unit is adapted for detecting a specific position of the exterior force applied to the outer surface of the body.

2. The apparatus according to claim 1, wherein the sensor unit measures the relative magnitude of the exterior force applied to the body.

3. The apparatus according to claim 1, wherein the sensor unit comprises two points of measurements placed at a maximum distance to each other of less than 0.3 cm.

4. The apparatus according to claim 1, wherein at least one sensor unit is a force sensor.

5. The apparatus according to claim 1, wherein the sensor unit measures a relative distributed force applied to said sensor unit from a muscle contraction.

6. The apparatus according to claim 1, wherein the sensor unit comprises at least 26 measurement points.

7. The apparatus according to claim 1, wherein the sensor array extends substantially along the whole length of the body.

8. The apparatus according to claim 1, wherein the sensor unit is adapted to detect a position of a force applied on the outer surface of the body.

9. The apparatus according to claim 1, wherein the body comprises a sensor support structure, the sensor array being mounted on said support structure.

10. The apparatus according to claim 9, wherein the support structure comprises a substantially flattened portion on to which the sensor array may be fitted so as the sensor unit is arranged on the substantially flattened portion.

11. The apparatus according to claim 1, wherein a muscle contraction is identified by analysing a distribution of total applied force over a number of measurement points.

12. The apparatus according to claim 2, comprising a visualizing means for visualising the position of the applied external force and/or the magnitude of the external force.

13. (canceled)

14. A method for visualizing pelvic floor muscle contractions, comprising the steps of:
a) using an apparatus comprising;
a body having a distal end, an opposite proximal end and an outer surface, the body extending between said ends,
a sensor array encompassed in the body and in the distal end of the body, the sensor array comprising a sensor unit, the sensor unit being adapted for detecting an exterior force applied to the outer surface of the body,
a micro-control unit adapted to receive sensor data from the sensor array,
a visualising means adapted for receiving sensor data from said micro-control unit,
b) inserting said apparatus into a body cavity of a user,
c) contracting at least one pelvic floor muscle of the user,
d) measuring the contraction on the outer surface of the body,
e) displaying data of the pelvic floor muscle contraction on the visualising means.

15. The method according to claim 14, wherein the data of the pelvic floor muscle contraction comprises;
the position of the contraction, and/or
the magnitude of the contraction.

16. The method according to claim 14, wherein the visualisation means is adapted to provide the user with feedback on the correctness of the muscle contraction, or the sensor unit is adapted for detecting a pressure profile of the exterior force applied to the outer surface of the body.

17. A method for visualizing the pelvic floor muscle contraction of a user, comprising: using a pelvic floor muscle training and profiling apparatus according to claim 1.

18. (canceled)

19. A method for producing a pelvic floor muscle training and profiling apparatus comprising the steps of:
a) providing a sensor support structure having a distal end, an opposite proximal end, the sensor support structure extending between said ends,
b) providing a micro-control unit and a sensor array comprising a sensor unit, said sensor array is adapted for detecting the position of an exterior force applied to the outer surface of the body
c) attaching the sensor array to the sensor support structure
d) providing power and communication lines between the micro-control unit for receiving sensor data and the sensor array to provide a sensor element.
e) encompassing the sensor element in an additional layer so as to provide the apparatus with a final structure.

20. The apparatus according to claim 1, wherein the sensor unit is adapted for detecting a pressure profile of the exterior force applied to the outer surface of the body.

21. The apparatus according to claim 1, wherein the sensor unit comprises at least 5 measurement points per cm.
